(19) United States
(12) Patent Application Publication
(10) Pub. No.: US 2005/0267448 A1
(43) Pub. Date: Dec. 1, 2005

(54) Endoscopic instrument

(75) Inventors: Ludwig Bonnet, Knittlingen (DE); Klaus-Peter Hipp, Grossvillars (DE); Thomas Ernst, Kurnbach (DE); Michael Fritz, Karlsruhe (DE)

Correspondence Address: AKIN GUMP STRAUSS HAUER & FELD L.L.P., ONE COMMERCE SQUARE, 2005 MARKET STREET, SUITE 2200, PHILADELPHIA, PA, 19103, US

(73) Assignee: Richard Wolf GmbH

(21) Appl. No.: 11/115,552

(22) Filed: Apr. 27, 2005

(30) Foreign Application Priority Data
May 17, 2004 (EP) 04 011 644.4
Jun. 29, 2004 (DE) 20 2004 010 223.4

(51) Int. Cl. A61B 17/00

(52) U.S. Cl. 606/1

(57) ABSTRACT

An endoscopic instrument, having a shank (2) envisaged for introduction into a body opening (8), has on its outer surface raised parts (10) lying at the corner points of a polygon, so that an essentially linear contact is formed between the shank (2) and the body.

### BACKGROUND OF THE INVENTION

The invention relates to an endoscopic instrument having a shank for introduction into a body opening.

An increasing number of diagnostic and therapeutic operations are nowadays carried out by minimal invasive techniques. For this purpose, the endoscopic instruments applied are pushed through body openings, which may be natural body openings, but also artificial channels created by puncture, to the application location inside the body. During the operation the outer wall of the endoscopes shank partially bears tightly against the body tissue of the opening channel, so that with operations having a longer duration, there exists the danger that the body tissue or the mucous membrane sticks to the shank and, with movements of the endoscope shank in the course of the operation, are torn away by this shank and traumatized.

For this reason it is usual to provide the contact surface between the shank and the surrounding body tissue with a layer of lubricant, in order thus to prevent the attachment of the body tissue to the shank. For this purpose, before the operation, the lubricant may be deposited onto the shank of the endoscopic instrument or it may be injected into the body opening concerned.

An endoscopic instrument is known from German published patent application DE 101 11 354 A1, which comprises supply channels by which the outer surface of the shank is provided with a lubricant capable of flowing. The supply of lubricant during the operation is effected in a continuous manner via a supply connection, which is arranged at the proximal end of the shank. The supply channels are arranged in the inside of the shank or are machined into the outer wall of the shank in the manner of a groove, wherein the shank has a tube-like covering (coating) which terminates the supply channels on the tissue side, and forms a contact surface to the body tissue. The contact surface of the shank with the surrounding tissue is connected to the supply channels by openings and is thus supplied with the lubricant. The disadvantage of this endoscopic instrument is that the reduction in friction is effected exclusively by the application of a lubricant, which must be supplied to the shank surface in the course of the operation. The supply channels and the supply connection require space and enlarge the outer diameter of the shank, which increases the danger of a traumatization of the surrounding tissue. Furthermore, a considerable manufacturing expense for forming the lubricant supply is required with this endoscopic instrument.

### BRIEF SUMMARY OF THE INVENTION

Against this background, it is an object of the invention to modify an endoscopic instrument, such that operations with this instrument may be performed in a manner that is gentler on the patient, and the endoscopic instrument is simple to realize with regard to manufacturing technology.

The endoscopic instrument according to the invention has a shank provided for introduction into a body opening. The outer contour of the shank cross section comprises raised parts, which lie at the corner points of a polygon, so that an essentially linear contact is formed between the shank and body. The polygonal cross-sectional surface of the shank may, for this purpose, have any number of corners.

The larger the contact surface between the shank and the surrounding body tissue, the larger is the danger of a drying-out and sticking of the body tissue to an endoscope shank. With the endoscopic instrument according to the invention, only the apex regions of the raised parts arranged on the shank contact the wall of the body channel, while the remaining shank surface has no contact with the body tissue. The linear contact surfaces which are formed by the raised parts of the endoscope shank with the surrounding body tissue are significantly smaller than the contact surfaces which are formed by the known cylindrical shanks of the same radial cross-sectional extent. Accordingly, the danger of a sticking or drying-out of the tissue is significantly reduced by the endoscopic instrument according to the invention. The shank accordingly has improved lubricant properties, so that in many cases, e.g., on guiding the shank into a urethra, it is not necessary to subject the shank surface to a lubricant, since the body's own moisture film alone, on account of the small contact region, is capable of preventing a drying-out of the tissue.

Despite this, it is advantageous to deposit a lubricant onto the shank of the endoscopic instrument before the operation. In this manner, a drying-out of the mucous membrane or the body tissue and thus a sticking during an operation may be almost ruled out.

The raised parts on the shank surface formed by the corners are relatively sharp-edged to a greater or lesser extent depending on the number of corners comprising the outer contour of the cross-sectional surface of the shank. Thus, reducing the number of corners leads to raised parts which converge in a more pointed manner, i.e., contact regions between the shank and the body cavity which are sharper edged. In order to prevent this, it is useful to design the raised parts on the surface of the shank in a rounded manner. In this manner, one prevents the shank movements in the body channel from causing a traumatization of the body tissue surrounding this channel.

It may also be advantageous if the surfaces between the raised parts are at least partially concavely curved in cross section. The surface regions of the shank lying between the raised parts are thus formed as rounded U-shaped recesses, which preferably extend in the longitudinal section of the shank. These recesses space large regions of the shank surface even further from the body tissue surrounding the channel in the direction of the longitudinal axis of the shank, which almost rules out their contact with the body tissue surrounding the channel. Furthermore, the recesses form ideal places for a lubricant to be deposited before the operation.

Preferably, the shank of the endoscopic instrument has on its outer periphery a wave-shaped profile, wherein the hills of the wave run essentially parallel to the longitudinal axis of the shank.

If the raised parts of the shank are formed in a rounded manner and the surfaces between the raised parts are curved in a concave manner, the radius of the corner rounding and the radius of curvature of the surfaces lying between the corners may be matched to one another, such that the outer contour of the shank cross section on the periphery is formed as a harmonic wave-shaped profile, wherein wave hills and valleys follow one another in a sinusoidally alternating manner. At the same time, over the entire periphery of the shank, the raised parts formed by the wave hills and the recesses formed by the wave valleys, have the same extension in the direction of the shank longitudinal axis. Thus, the radial cross-sectional extension of the shank over its periphery varies periodically between a maximum value at the apexes of the wave hills and a minimum value at the nadirs of the wave valleys.

Since the shank cross section is designed with the above-described shape over the entire length of the shank, there results at the shank surface a periodically changing sequence of wave hills, which form ridge-like raised parts, and wave valleys, which form depressions indented toward the shank longitudinal axis, this sequence extending along the direction of the shank longitudinal axis.

In this manner, the apex lines of the wave hills form the contact region between the shank and the surrounding body tissue. If during an operation the endoscopic instrument moves in a body channel in the direction of the shank longitudinal axis, then only narrow linear regions of the inner wall of the body channel come into contact with the surface of the shank. Also, with rotational movement of the shank in the body channel, the endoscopic instrument according to the invention has significantly improved sliding (lubrication) properties over a known shank formation. A wave-shaped profile of the shank is also particularly favorable with regard to manufacturing technology, since such a wave-shaped profile may be formed in a simple manner by deformation of the shank wall, for example by drawing, pressing or embossing a cylindrical tube.

The essentially linear contact regions of the shank according to the invention are, however, not limited to the previously mentioned wave-shaped profile running in an axially parallel manner, but may also be designed differently depending on the requirements. Thus, the raised parts over the periphery of the shank may, for example, also be arranged in a twisted manner, so that a helical arrangement of the linear contact regions on the shank results. Such a shank design is particularly advantageous for a combined axial and rotational movement, i.e., when a helical movement of the shank within the body channel is envisaged.

Preferably, about 5 to 20 raised parts are distributed on the surface of the shank around the periphery for the linear contact region with the body tissue surrounding the channel. The number of raised parts is dependent on the size of the shank cross section, so that shanks with a larger radial cross-sectional dimension may have more raised parts on their outer periphery than shanks with a smaller radial cross-sectional dimension. The outer radial shank dimension with the instrument according to the invention preferably lies in a region of about 5 to 10 mm, wherein the size of the cross-sectional surface is adapted, as an initial matter, to the operation conditions, i.e., to the dimensions of the corresponding body channel.

In a further embodiment, the shank of the endoscopic instrument comprises linear contact regions which are arranged on the surface of the shank in the manner of a grid-net. For example, the surfaces between the raised parts are not continuously indented concavely in the direction of the shaft longitudinal axis, but as a series of concavely curved pockets, which are delimited by narrow webs which are not indented. The grid structure formed in this manner comprises a multitude of regions, which are completely enclosed by linear raised parts. The apex lines of the raised parts form the contact regions to the body tissue, while the enclosed regions are spaced from the body tissue and thus not may come into contact with the tissue. If the shank of the endoscopic instrument is subjected to a lubricant before the operation, the deeper lying regions form closed depots from which the lubricant may not flow away.

It may be advantageous to interrupt the contact regions in the direction of their longitudinal extension. Thus, long linear contact regions may be divided into a series of contact regions, which are spaced from one another and which extend in a common extension direction. The contact surface with the surrounding body tissue is thereby further reduced.

With the above described embodiments it is advantageously possible to design the shank surface such that the contact regions with the surrounding body tissue are reduced by about 55 to 75% with respect to a cylindrical shank with the same radial cross-sectional dimension. Accordingly, the endoscope shank does not contact about 55 to 75% of the surrounding body tissue. Since with a movement of the shank, the loading of the surrounding tissue by sliding friction with the shank is directly proportional to the size of the contact surface with the shank, and this loading is reduced equally with regard to its share. The result of this is that the tissue is loaded to a significantly lower extent when moving the endoscopic instrument.

The height of the raised parts which form the contact regions with the body tissue advantageously have a ratio of about 1:10 to 1:20 to the smallest enveloping diameter, i.e., to the diameter of an imaginary enveloping circle running through the apexes of the recesses. In this manner, the improved contact conditions mentioned above are created without using instruments having a hollow shank with the inner space of the shank or its inner cross section being narrowed.

The formation of the region raised linearly with respect to the remaining shank, as with the initially described wave-shaped profile, may be effected either by deformation of a cylindrical hollow shank or also by a targeted material deposition onto a cylindrical shank. For this purpose, the material deposition may be effected by welding or another suitable deposition method. Conversely, the raised parts may, however, also be produced by material removal methods, such as spark erosion in those regions next to the raised linear regions. Here too, for producing a grid- or net-like structure, a cylindrical shank may be covered with a net-shaped enveloping flexible tubing.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a schematic cross-sectional representation of a shank of an endoscopic instrument according to the invention, located in a body channel, with a polygonal shank cross section;

FIG. 2 is a schematic cross sectional representation of a shank of an alternative embodiment of an endoscopic instrument according to the invention, located in a body channel, with the shank having concavely curved peripheral regions; and

FIG. 3 is a schematic cross-sectional representation of a further embodiment of a shank of an endoscopic instrument according to the invention, located in the body channel, the shank having a wave-like outer cross-sectional contour.

### DETAILED DESCRIPTION OF THE INVENTION

With the shanks 2, 2′ and 2″ represented in the Figures, there are shown embodiments of (any) hollow shank of an endoscopic instrument (not shown here). Each shank comprises a casing tube 4, 4′ and 4″ in whose inner space 6, 6′ and 6″ working tools exiting the distal shank end (not shown in the drawing) are guided or further components are arranged.

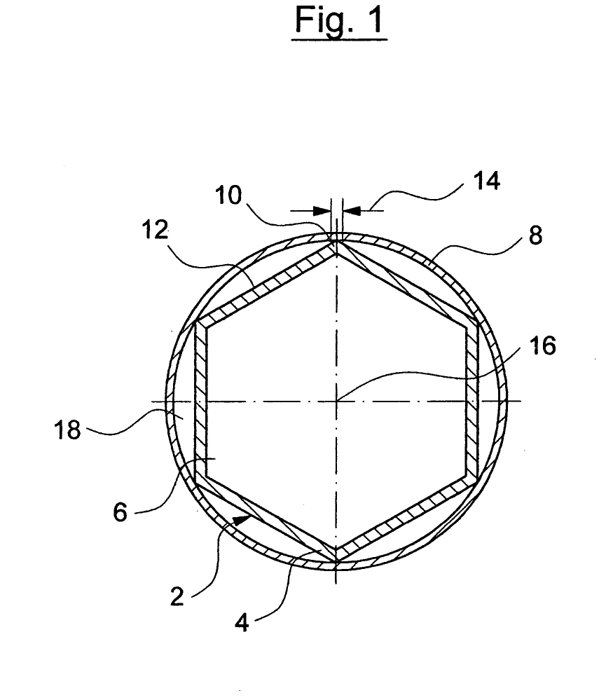

The shank 2 represented in FIG. 1 is located in a body channel 8 and has a polygonal outer cross-sectional contour with six corners 10 and plane surface segments 12 lying therebetween. The number of corners 10 in the shown embodiment is six. However, the shank cross section may have any number of corners 10 depending on the particular application, wherein about 5 to 20-cornered cross-sectional contours are preferred. The corners 10 form raised parts having contact regions 14 with the surrounding body channel 8, and extend on the shank surface in the direction of its longitudinal axis 16. In this manner, large regions of the surface segments 12 are indented from the body channel 8 and form intermediate spaces 18 between the body channel 8 and the surface of the shank 2, so that in this region of the shank surface there exists no contact with the body tissue. In order to prevent a traumatization of the body tissue of the body channel 8, the corners 10 are designed in a rounded manner.

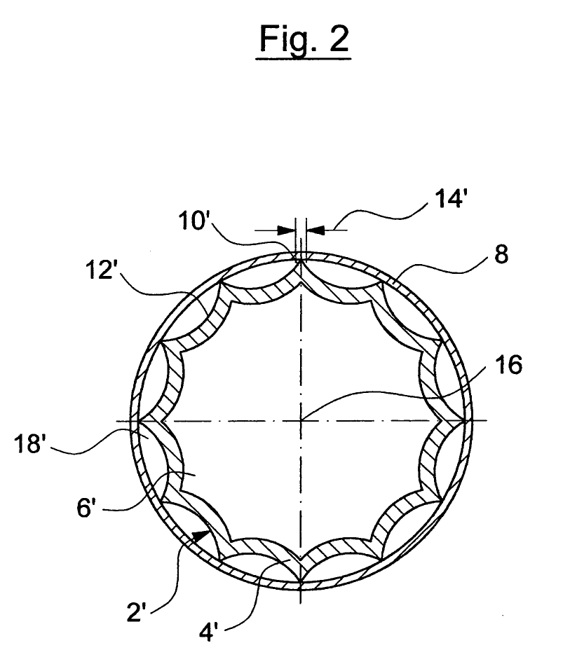

FIG. 2 shows the cross section of a shank 2′ having a 12-cornered outer contour. The surface segments 12′ lying between the corners 10′ are concave, i.e., are curved inwards toward the shank longitudinal axis 16. Due to this curvature, the corners 10′ form raised parts projecting outwardly even more significantly compared to the raised parts represented in FIG. 1. Thus, the shank surface has over its periphery a uniformly, distributed succession of ridge-like raised parts and U-shaped depressions, which run in the direction of the longitudinal axis 16 of the shank 2′. At the same time, linear contact regions 14′ of the raised parts contact the wall of the body channel 8. The surface segments 12′ lying between the raised parts or corners 10′ are spaced even further from the wall of the body channel, due to their concave curvature. The intermediate spaces 18′ which are formed by the body channel 8 and the surface segments 12′ are correspondingly large, and form suitable depots for a lubricant to be deposited before the application of the endoscopic instrument to the body channel.

Although not shown in FIG. 2, it is recommended with the embodiment introduced in FIG. 2 to shape the corners 10′ in a rounded manner, since due to the curvature of the surface segments 12′ the corners 10′ forming these corners 10′ converge in a comparatively pointed manner. Accordingly, these corners 10′ may form sharp-edged cutting edges on the surface of the shank 2′, by which the body tissue surrounding the body channel 8 could be traumatized.

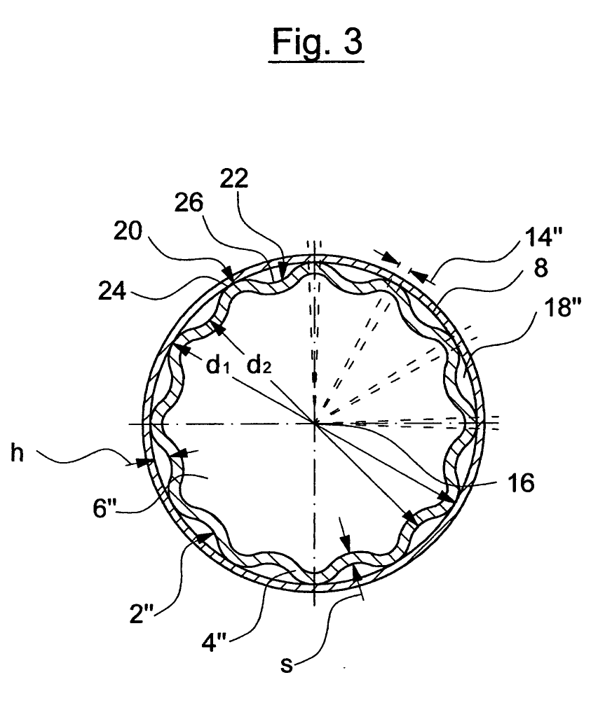

A particularly preferred embodiment of the shank 2′ therefore envisages rounding off the corners 10′, so that the rounding of the corners merges into the concave curvature of the surface segments 12′ in a harmonic manner. Such a rounding of corners results, for example, in the outer cross-sectional contour of the shank 2′ having the wave shape shown in FIG. 3.

In FIG. 3 the wall of the casing tube 4″ is designed wave-like in cross section, so that sinusoidally changing wave hills 20 and wave valleys 22 follow one another. Accordingly, the radial cross-sectional extension of the shank 2″ changes periodically and grows continuously to a maximum value d1 at the apexes 24 of the wave hills 20 and subsequently reduces in a continuous manner to a minimum value d2 at the nadirs 26 of the wave valleys. The distance h between the apexes 24 of the wave hills 20 and the nadirs of the wave valleys 22 in the radial direction is roughly as large as the wall thickness s of the casing tube 4″ and preferably has a ratio of about 1:10 to 1:20 to the minimum radial cross-sectional extension d2 of the casing tube 4″. Hence, the wave valleys 22 directed toward the shank longitudinal axis 16 do not narrow the cross section of the inner space 6″ of the casing tube 4″ so much that the positioning of the working tools of the endoscopic instrument guided in this inner space is hindered. Respective apexes 24 and nadirs 26, which are adjacent in the peripheral direction, have the same spacing from one another over the whole periphery of the shank 2″.

The wave hills 20 and wave valleys 22 on the outer surface of the shank 2″ form, respectively, 12 raised parts 20 and recesses 22. These raised parts 20 and recesses 22 run linearly over the whole length of the shank 2″ and specifically parallel to the shank axis 16. However, it is also possible to profile only partial regions of the shank 2″ in a wave-like manner, for example to design the proximal end region of the shank 2″ in a cylindrical or prism-shaped manner.

The apex regions of the raised parts 20 form the contact surface 14″ with the surrounding body channel 8, while the recesses 22 and large regions of the raised parts 20 do not come into contact with the body tissue, but form intermediate spaces 18″ between the outer wall of the shank 2″ and the surrounding body channel 8. These spaces may serve as depots for a lubricant to be deposited before the application of the endoscopic instrument.

Although an endoscopic instrument with a hollow shank is shown in the Figures, instruments may also form the shank surface with a solid shank as described above.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. An endoscopic instrument having a shank (2, 2′, 2″) for introduction into a body opening (8), wherein an outer cross-sectional contour of the shank (2, 2′, 2″) comprises raised parts lying at corner points of a polygon, such that regions of essentially linear contact (14, 14′, 14″) are formed between the shank (2, 2′, 2″) and the body (8).

2. The endoscopic instrument according to claim 1, wherein the raised parts of the shank (2, 2′, 2″) are rounded off.

3. The endoscopic instrument according to claim 1, wherein surfaces (12, 12′) of the shank (2, 2′, 2′) between the raised parts are at least partially concavely curved in cross section.

4. The endoscopic instrument according to claim 1, wherein the shank (2″) has on its outer periphery a wave-shaped profile, wherein the wave hills (20) run essentially parallel to a longitudinal axis (16) of the shank (2″).

5. The endoscopic instrument according to claim 2, wherein an outer cross-sectional contour of the shank (2, 2′, 2″) has about 5 to 20 raised parts.

6. The endoscopic instrument according to claim 1, wherein the shank comprises linear contact regions (14, 14′, 14″) arranged on a shank outer surface in a manner of a grid-net.

7. The endoscopic instrument according to claim 1, wherein the contact regions (14, 14′, 14″) are interrupted in a direction of their longitudinal extension.

8. The endoscopic instrument according to claim 1, wherein contact of the contact regions (14, 14′, 14″) with the surrounding body (8) is reduced by about 55 to 75% compared to a circular shank of a same radial cross-sectional extension.

9. The endoscopic instrument according to claim 1, wherein the shank has maximum radial cross-sectional extensions of about 5 to 10 mm.

10. The endoscopic instrument according to claim 1, wherein a height (h) of the raised parts (20) has a ratio of about 1:10 to 1:20 with respect to a minimum enveloping diameter (d2) of the shank (2″).
